(19) United States
(12) Patent Application Publication
(10) Pub. No.: US 2016/0201067 A1
(43) Pub. Date: Jul. 14, 2016

(54) Composition for Enhancing Transgene Expression in Eukaryotic Cells and Method for Enhancing Production of a Target Protein Encoded by a Transgene

(71) Applicants: Rustam R. ATAULLAKHANOV, Moscow (RU); SILEZIA GROUP PTE. LTD, Singapore (SG)

(72) Inventors: Rustam R. ATAULLAKHANOV, Moscow (RU); Ravshan I. ATAULLAKHANOV, Moscow (RU); Aleksandr V. BAGAEV, Nizhnekamsk (RU); Aleksandr L. GINTSBURG, Moscow (RU); Denis Ju. LOGUNOV, Moscow (RU); Boris S. NARODITSKY, Moscow (RU); Aleksey V. PICHUGIN, Moscow (RU); Elena S. SEDOVA, Shikhany (RU); Irina L. TUTYKHINA, Moscow (RU); Amir I. TUKHVATULIN, Moscow (RU); Rakhim M. KHAITOV, Moscow (RU); Maksim M. SHMAROV, Moscow (RU)

(21) Appl. No.: 15/018,459

(22) Filed: Feb. 8, 2016

Related U.S. Application Data
(63) Continuation of application No. PCT/RU2013/000997, filed on Nov. 8, 2013.

(30) Foreign Application Priority Data
Aug. 7, 2013 (RU) 2013136874

(51) Int. Cl. C12N 15/67; A61K 31/7028; A61K 38/16; A61K 31/739; A61K 31/715; C07K 14/005; C07K 14/435; C07K 14/195; C12N 9/16; C07K 14/525; C12N 15/85; A61K 31/713; A61K 31/4745

(52) U.S. Cl. CPC C12N 15/67; A61K 31/713; A61K 31/7028; A61K 38/16; A61K 31/739; A61K 31/715; A61K 38/164; A61K 31/4745; C07K 14/43504; C07K 14/195; C12N 9/16; C07K 14/525; C12N 15/85; C07K 14/005; C12Y 301/03001

(57) ABSTRACT

Production of DNA vectors with the inserted gene of a target protein and the production of recombinant protein in eukaryotic cell cultures is disclosed. A composition for the intensive production of target protein in eukaryotic cells comprises a DNA vector with the inserted gene of a target protein and an agonist of cell receptors belonging to the pattern recognition receptor (PRR) family selected from the following agonists: TLR2, or TLR4, or TLR5, or TLR7, or TLR8, or TLR9, or NOD1 receptor, or NOD2 receptor, used in an optimal ratio. TLR2 is a lipoteichoic acid. TLR2 is a lipopeptide. TLR4 can be either bacterial lipopolysaccharide or acidic peptidoglycan (APG) having a molecular weight of 1200-40000 kDa. TLR5 is flagellin TLR7 is either imiquimod or CL097, an imidazoquinoline derivative. TLR8 is either imiquimod or CL097, an imidazoquinoline derivative. TLR9 is either oligonucleotide CpG ODN 1826 or oligonucleotide CpG ODN 2006.

### RELATED APPLICATIONS

This Application is a Continuation application of International Application PCT/RU2013/000997, filed on Nov. 8, 2013, which in turn claims priority to Russian Patent Applications No. RU2013136874, filed Aug. 7, 2013, both of which are incorporated herein by reference in their entirety.

### FIELD OF THE INVENTION

The present invention relates to the field of biotechnology and medicine. More particularly, the invention concerns the production of DNA vectors with the inserted gene encoding target protein, and production of recombinant proteins in eukaryotic cell cultures, and manufacturing of modified cells for cellular therapy, and performing cell and gene therapies in humans and animals.

### BACKGROUND OF THE INVENTION

DNA vectors are used in various fields of biology for the delivery of exogenous genetic material to cells and the expression of exogenous genes. They are used as molecular biology tools in both in vitro studies, e.g., for the study of functions of certain genes, and in vivo for the transfer of genetic information into host cells for gene therapy or vaccination purposes. DNA vectors are also applied for modification of cells which are further used either for the production of a target protein or as tools for gene therapy.

A target gene expression level is a key characteristic of DNA vectors in all of the above mentioned applications. Therefore, enhancing expression of a transgene delivered to cells is a special task during development of DNA vector-based systems. This affects the level of transgene production during gene therapy or the level of immune response to genetic immunization. Moreover, with the enhanced transgene expression, the dose of vector administered in vitro or in vivo may be reduced.

The level of target gene expression can be enhanced by two means. The first one uses incorporation of different regulatory elements, namely, promoters, polyadenylation signals, introns, exons, and 5′- and 3′-nontranslated elements such as PARS, IRES, and WPRE into a DNA vector expression cassette [Dorokhov Y L, Skulachev M V, Ivanov P A, Zvereva S D, Tjulkina L G, Merits A, Gleba Y Y, Hohn T, Atabekov J G. Polypurine (A)-rich sequences promote cross-kingdom conservation of internal ribosome entry//Proc. Natl. Acad. Sci. USA, 2002, v. 99, p. 5301-5306. Lee Y B, Glover C P, Cosgrave A S, Bienemann A, Uney J B. Optimizing regulatable gene expression using adenoviral vectors.//Exp Physiol., 2005 January; 90(1):33-37. Li Z L, Tian P X, Xue W J, Wu J. Co-expression of sCD40LIg and CTLA4Ig mediated by adenovirus prolonged mouse skin allograft survival.—J Zhejiang Univ Sci B, 2006 June; 7(6):436-44. US 20080241883 A1 Recombinant expression vector elements (rEVEs) for enhancing expression of recombinant proteins in host cells. WO2008000445 Expression vector(s) for enhanced expression of a protein of interest in eukaryotic or prokaryotic host cells].

The second means is a treatment of cells transduced with DNA vector using external molecular agents such as butyrate and trichostatin A [Siavoshian S., J-P Segain, M. Kornprobst, C. Bonnet, C. Cherbut, J-P. Galmiche, H. M. Blottiere. Butyrate and trichostatin A effects on the proliferation/differentiation of human intestinal epithelial cells: induction of cyclin D3 and p21 expression.//Gut, 2000; 46:507-514].

Replication-defective DNA vectors, e.g., recombinant pseudoviral particles cannot replicate [U56019978 Replication-defective adenovirus human type 5 recombinant as a vaccine carrier]; therefore, the effectiveness of target transgene expression depends on the ability of viral particles to transduce cells and on the effectiveness of further production of target protein.

The in vivo effectiveness can be increased either by increasing the dose of administered viruses [Tutykhina I L, Sedova E S, Gribova I Y, Ivanova T I, Vasilev L A, Rutovskaya M V, Lysenko A A, Shmarov M M, Logunov D Y, Naroditsky B S, Tillib S V, Gintsburg A L. Passive immunization with a recombinant adenovirus expressing an HA (H5)-specific single-domain antibody protects mice from lethal influenza infection.//Antiviral Res., 2013 March; 97(3):318-28] or by increasing the quality of administered viral particles [RU2443779 Method for obtaining recombinant adenovirus preparation characterized by a decreased ratio of physical and infectious viral particles and a gene therapy drug obtained using the said method].

There is a known invention [US20080311095 Methods and compositions for increased transgene expression] proposing a method for increasing transgene expression in human T cells by their preliminary in vitro activation with anti-CD3/anti-CD28 antibodies. This invention was selected as a prototype. The apparent disadvantages of the prototype are as follows: (1) This invention can be used only for the activation of T cells and cannot be used for the activation of other types of cells, because the CD3 receptor is synthesized only by T cells; (2) the type of T cell activation proposed in the prototype can be conducted only in vitro; and (3) after the activation with anti-CD3/anti-CD28 antibodies, as proposed in the prototype, all T cells will actively proliferate (polyclonal activation). These polyclonally in vitro activated T cells cannot be administered to human subjects or animals, because this may lead to severe autoimmune, systemic inflammatory, lymphoproliferative processes and complete alteration of normal functions of the immune system, with a very high risk of lethal outcome.

### SUMMARY OF THE INVENTION

The task of the present invention was to develop a composition for enhancing transgene expression in eukaryotic cells and a method for enhancing production of a target protein encoded by a transgene and to provide an opportunity of using the said composition and method both in cell culture in vitro and in a living body (in vivo), without harmful effects on health and life.

To achieve this task, a composition for the intensive production of target protein in eukaryotic cells has been developed, which comprises a DNA vector with the inserted target protein gene and an agonist of cell receptors belonging to the family of pattern recognition receptors (PRR), which is selected from the following agonists: TLR2 agonists, or TLR4 agonists, or TLR5 agonists, or TLR7 agonists, or TLR8 agonists, or TLR5 agonists, or NOD1 receptor agonists, or NOD2 receptor agonists, used in optimal ratio. In this embodiment, lipoteichoic acid is used as a TLR2 agonist or lipopeptide is used as a TLR2 agonist. As a TLR4 agonist, either bacterial lipopolysaccharide or acidic peptidoglycan (APG) having a molecular weight of 1200-40000 kDa is used. As a TLR5 agonist, flagellin is used. As a TLR7 agonist, either imiquimod or CL097, an imidazoquinoline derivative, is used. As a TLR8 agonist, either imiquimod or CL097, an imidazoquinoline derivative, is used. As a TLR9 agonist, either oligonucleotide CpG ODN 1826 or oligonucleotide CpG ODN 2006 is used. As NOD1 receptor agonists, C12-iE-DAP-Lauroyl-g-D-Glu-D-mDAP and Lauroyl-g-D-Glu-L-mDAP, synthetic fragments of bacterial peptidoglycan, are used. As a NOD2 receptor agonist, L18-MDP, a derivative of muramyl dipeptide, specifically, the fragment of bacterial peptidoglycan, is used. As a PRR agonist, a pharmaceutical drug in an effective dose may be used. As a PRR agonist, Immunomax (Reg. No. 001919/02) is used. As a PRR agonist, Pyrogenalum (Reg. No. 003478/0) may be also used. As a PRR agonist, Licopid (Reg No. 001438) may be also used. As a DNA vector, replication-defective recombinant human adenovirus (serotype 5) nanoparticles are used. A DNA vector with the inserted target gene encoding a secretory protein is used, or a DNA vector with the inserted target gene encoding a cytoplasmic protein is used, or a DNA vector with the inserted target gene encoding a membrane protein is used.

The claimed composition is used in the method for enhancing production of a target protein encoded by a transgene in eukaryotic cells transduced with DNA vectors. In this embodiment, the enhanced production of a target protein is achieved in cultures of eukaryotic cells in vitro. The enhanced production of a target protein is also achieved in vivo, in a living body. In this embodiment, eukaryotic cells are obtained from a mouse, or eukaryotic cells are obtained from a human body.

A composition for the intensive production of a target protein in eukaryotic cells may be also used, which comprises a DNA vector with inserted gene of the target protein and an agonist of cell receptors belonging to the cytokine receptor family, used in optimal ratio. In this case, tumor necrosis factor (TNF) is used as an agonist of cytokine receptors. The method is proposed here for enhancing production of target protein in eukaryotic cells which are transduced with the above mentioned composition comprising DNA vectors and an agonist of cytokine receptors.

Thus, the technical result, i.e. enhanced production of a target transgenic protein has been achieved by using agonists of PRR and cytokine receptors.

The specific feature of the present invention is the use of agonists of PRRs from families of TLR and NOD receptors, and agonists of cytokine receptors (TNF).

The proposed invention provides the following advantages: (1) the effect is achieved both in vitro and in vivo; (2) the effect can be achieved in any type of cells that can express the DNA vector and bear functionally active PRRs; and (3) the proposed method for enhancing transgene expression does not lead to polyclonal proliferation and to other pathological effects harmful to human subjects and animals, and pharmaceutical grade PRR agonists may be used for enhancing transgene expression according to the proposed method.

The present invention proposes a composition for enhancing transgene expression in eukaryotic cells and a method for enhancing production of a target protein encoded by a transgene.

The task achieved by the present invention is a significant enhancement of the target transgene expression and target protein production in eukaryotic cells both in cell culture in vitro and in human subjects and animals in vivo.

It is known that gene transcription and protein production in eukaryotic cells may be significantly changed under the effect of external signals which can be classified into several basic types, which are as follows:

a) contact between the eukaryotic cell and pathogens or pathogen components, the so-called pathogen associated molecular patterns (PAMPs);

b) contact between the eukaryotic cell and damaged cells or their components, the so-called damage-associated molecular patterns (DAMPs);

c) cytokines and danger signals, which are chemical regulatory signals for cell-to-cell communications;

d) contact interactions of eukaryotic cells with each other; and

e) contact between the cell and extracellular matrix polymers.

In the present invention, the task of enhancing transgene expression and target protein production is achieved by the activation of transduced cells with PAMPs or DAMPs acting on cells via PRRs, or with cytokines acting on cells via cytokine receptors, or by simulation of these actions by using agonistic compounds acting on eukaryotic cells via PRRs or cytokine receptors.

In the present invention, for enhancing transgene expression and target protein production in eukaryotic cells transduced by a DNA vector with the inserted transgene, substances activating cellular metabolism via PRRs or cytokine receptors are used. A use of proposed combination of DNA vectors with said types of activators of cellular metabolism results in 2- to 10-fold increased production of target proteins related to cytoplasmic, membrane, or secretory proteins.

The description of the invention proves that, in the eukaryotic cells transduced by the DNA vector with the inserted transgene, the enhancement of transgene expression and target protein production occurs by using agonists acting via PRRs or cytokine receptors, which can be effectively implemented in eukaryotic cells both in vitro and in vivo.

The enhancement effect is observed in cells of both animals (mouse) and human subjects. The enhancement is observed in cell types that can be transduced by and express this DNA vector and bear functionally active PRRs.

Transgene expression enhancers proposed in the present invention can be used in vivo, as was shown in laboratory animals, and it was also shown that pharmaceutical drugs may be used as PRR agonists.

### BRIEF DESCRIPTION OF THE DRAWINGS

FIG. 1A is a histogram illustrating production of SEAP reporter protein by HEK-Blue-TLR4 cells in response to acidic peptidoglycan confirms that this ligand activates NF-kB via TLR4. The activity of NF-κB was estimated by the expression of NF-kB dependent SEAP reporter gene in HEK-Blue cells bearing no TLRs (null) and in cells expressing one of the following receptors: TLR2, TLR3, TLR4, TLR5, TLR7, TL8, or TLR9. Cells were incubated for 18 h in the absence (negative control, □) or in the presence (▪) of APG (5 μg/ml). The standard agonist ( [symbol] ) was used as a positive control for each type of cells: TNF-α (10 ng/ml) for null cells, lipopeptide (1 μg/ml) for TLR2 cells, poly I:C (10 μg/ml) for TLR3 cells, LPS (1 μg/ml) for TLR4 cells, flagellin (1 μg/ml) for TLR5 cells, imiquimod (1 μg/ml) for TLR7 cells, CL097 (1 μg/ml) for TLR8 cells, and ODN 2007 (10 μg/ml) for TLR9 cells. Results are given as a fold increase in the production of NF-kB dependent SEAP reporter protein compared to control cells (without an activator). Mean values obtained in three independent experiments are shown. The conclusion is that acidic peptidoglycan (APG, Russian Patent no. 2195308) activates NF-kB via TLR4;

FIG. 1B is a graph illustrating production of NF-kB dependent SEAP reporter protein by HEK-Blue-TLR4 cells in response to APG. Dependence on the concentration of APG. HEK-Blue-TLR4 cells were incubated for 18 h in the presence of indicated concentrations of APG. Intact HEK-Blue-TLR4 cells as well as HEK-Blue-TLR-null cells were used as negative controls. Results are given as a fold increase in the production of NF-kB dependent SEAP reporter protein compared to control cells (without an activator). Mean values of data obtained in three independent experiments are shown;

FIG. 2A is a histogram illustrating identification of a cellular receptor for a pharmaceutical drug Immunomax (Reg. no. 001919/02), TLR-null cells; HEK Blue TLR (InvivoGen) cells expressing one of TLRs (TLR2, TLR3, TLR4, TLR5, TLR7, TLR8, or TLR9) or bearing no TLRs (null cells) were used to identify cellular receptor for a pharmaceutical drug Immunomax (Reg. no. 001919/02);

FIG. 2B is a histogram illustrating identification of a cellular receptor for a pharmaceutical drug Immunomax (Reg. no. 001919/02), cells with TLR2 and co-receptors CD14; HEK Blue TLR (InvivoGen) cells expressing one of TLRs (TLR2, TLR3, TLR4, TLR5, TLR7, TLR8, or TLR9) or bearing no TLRs (null cells) were used to identify cellular receptor for a pharmaceutical drug Immunomax (Reg. no. 001919/02);

FIG. 2C is a histogram illustrating identification of a cellular receptor for a pharmaceutical drug Immunomax (Reg. no. 001919/02), cells with TLR3; HEK Blue TLR (InvivoGen) cells expressing one of TLRs (TLR2, TLR3, TLR4, TLR5, TLR7, TLR8, or TLR9) or bearing no TLRs (null cells) were used to identify cellular receptor for a pharmaceutical drug Immunomax (Reg. no. 001919/02);

FIG. 2D is a histogram illustrating identification of a cellular receptor for a pharmaceutical drug Immunomax (Reg. no. 001919/02), cells with TLR4 and co-receptors CD14 and MD2; HEK Blue TLR (InvivoGen) cells expressing one of TLRs (TLR2, TLR3, TLR4, TLR5, TLR7, TLR8, or TLR9) or bearing no TLRs (null cells) were used to identify cellular receptor for a pharmaceutical drug Immunomax (Reg. no. 001919/02);

FIG. 2E is a histogram illustrating identification of a cellular receptor for a pharmaceutical drug Immunomax (Reg. no. 001919/02), cells with TLR5; HEK Blue TLR (InvivoGen) cells expressing one of TLRs (TLR2, TLR3, TLR4, TLR5, TLR7, TLR8, or TLR9) or bearing no TLRs (null cells) were used to identify cellular receptor for a pharmaceutical drug Immunomax (Reg. no. 001919/02);

FIG. 2F is a histogram illustrating identification of a cellular receptor for a pharmaceutical drug Immunomax (Reg. no. 001919/02), cells with TLR7; HEK Blue TLR (InvivoGen) cells expressing one of TLRs (TLR2, TLR3, TLR4, TLR5, TLR7, TLR8, or TLR9) or bearing no TLRs (null cells) were used to identify cellular receptor for a pharmaceutical drug Immunomax (Reg. no. 001919/02);

FIG. 2G is a histogram illustrating identification of a cellular receptor for a pharmaceutical drug Immunomax (Reg. no. 001919/02), cells with TLR8; HEK Blue TLR (InvivoGen) cells expressing one of TLRs (TLR2, TLR3, TLR4, TLR5, TLR7, TLR8, or TLR9) or bearing no TLRs (null cells) were used to identify cellular receptor for a pharmaceutical drug Immunomax (Reg. no. 001919/02);

FIG. 2H is a histogram illustrating identification of a cellular receptor for a pharmaceutical drug Immunomax (Reg. no. 001919/02), cells with TLR9; HEK Blue TLR (InvivoGen) cells expressing one of TLRs (TLR2, TLR3, TLR4, TLR5, TLR7, TLR8, or TLR9) or bearing no TLRs (null cells) were used to identify cellular receptor for a pharmaceutical drug Immunomax (Reg. no. 001919/02); Cells of all of the above lines were incubated in a complete culture medium in vitro for 18 h at 37° C. and 5% CO2 in the absence or presence of Immunomax (from 0.02 to 15 μg/ml). The standard agonist was used as a positive control for each type of cells: (A) TNF-α (10 ng/ml) for null cells, (B) lipopeptide (1 μg/ml) for TLR2 cells, (C) poly I:C (10 μg/ml) for TLR3 cells, (D) LPS (1 μg/ml) for TLR4 cells, (E) flagellin (1 μg/ml) for TLR5 cells, (F) imiquimod (1 μg/ml) for TLR7 cells, (G) CL097 (1 μg/ml) for TLR8 cells, and (H) ODN 2007 (10 μg/ml) for TLR9 cells. Activation of TLRs (TNF-α receptors, in case of TLR-null cells as control cells) was estimated by activation of production of SEAP reporter protein, the gene of which is under the control of an NF-kB dependent promoter in all types of cells used in this work. Results are given as a fold increase in the production of NF-kB dependent SEAP reporter protein compared to control cells (without an activator). Mean values obtained in three independent experiments are shown.

FIG. 3A is an illustration of the analysis of PRR agonist-induced enhancement of Ad-GFP transgene expression in peritoneal macrophages by using confocal microscopy and flow cytometry techniques: peritoneal macrophages were transduced with Ad-GFP (2×10⁷ PFU/ml) in the absence (left photo) or in the presence (right photo) of TLR4 agonist (APG, 10 μg/ml);

FIG. 3B and FIG. 3C are an illustration of the consecutive steps of live macrophages gating during cytometry, according to their light scattering and staining with DAPI, a DNA-specific dye;

FIG. 3D and FIG. 3E are graphs illustrating analysis of the intensity of GFP fluorescence of peritoneal macrophages transduced with Ad-GFP (2×10⁷ PFU/ml) in the absence (D) or in the presence (E) of TLR7/8 agonist CL097 (2.5 μg/ml). Cell cultures were incubated for 4 days at 37° C. and 5% CO2. After the end of incubation, cells were collected by washing with a cold Versene solution, and the percentage and absolute number of cells having green fluorescence and the intensity of cell fluorescence were determined using a FACSAria II flow cytometer. The absolute count of cells was normalized by calibration beads containing a known concentration of microspheres (CountBright Invitrogen);

FIGS. 4A, 4B, and 4C are histograms illustrating enhancement of expression of the cytoplasmic transgene protein (GFP) in peritoneal macrophages in vitro by using pharmaceutical agonists of PRRs. Mouse peritoneal macrophages were transduced with Ad-GFP (2×10⁵ PFU) without additional activation (control) or with activation of cells using one of the pharmaceutical agonists of PRRs, specifically, Immunomax (Reg. no. 001919/02-171011), or Pyrogenalum (Reg. no. 003478/0), or Licopid (Reg. no. 001438). Cells were incubated for 2 days in a complete culture medium at 37° C. and 5% CO2. After the end of incubation, the cells were collected by washing with a cold Versene solution, and (FIG. 4A) the normalized number of macrophages having green fluorescence and (FIG. 4B) the normalized mean intensity of fluorescence were determined using a FACSAria II flow cytometer. (FIG. 4C) The integral fluorescence of macrophages was calculated as the product of the number of fluorescent cells and the mean intensity of cell fluorescence. Data were normalized by control values (Ad-GFP without additional cell activation).

### DETAILED DESCRIPTION OF THE PREFERRED EMBODIMENTS

The task solved in the present invention is a significant enhancement of the target transgene expression and target protein production in eukaryotic cells both in cell culture in vitro and in humans and animals in vivo.

In the present invention, replication-defective recombinant adenovirus nanoparticles (RDRANs) encoding cytoplasmic, membrane, or secretory proteins were used as DNA vectors. A DNA vector was expressed in the following eukaryotic cell cultures: mouse primary spleen, bone marrow, and peritoneal cells, mouse bone marrow cells differentiated in vitro in the presence of granulocyte-macrophage colony-stimulating factor (GM-CSF), and the fraction of human blood mononuclear cells. Transgene expression was analyzed depending on the vector used. For RDRANs with inserted gene encoding green fluorescent protein (Ad-GFP), the expression was analyzed using fluorescent and confocal microscopy, and flow cytofluorometry. For RDRANs with inserted hemagglutinin gene of influenza H1N1 (Ad-HA1), H3N2 (Ad-HA3) or B (Ad-HA-B) viruses, the target protein production was analyzed using cytofluorometry of cells stained with monoclonal antibody specifically bound to HA protein. The expression of Ad-SEAP, the RDRANs with inserted gene of secreted embryonic alkaline phosphatase (SEAP), was estimated by secretion of target SEAP protein, the concentration of which was measured using a colorimetric assay for SEAP enzymatic activity.

The enhancement of expression was achieved by using PRR agonists. According to current understanding of PRRs, human and animal cells possess several families of PAMP- and DAMP-recognizing receptors that enable cells not only to detect and respond to contacts with any microbial pathogen or its components but also to recognize damage of host tissues and adequately respond to this damage.

PRRs include Toll-like receptors (TLRs), NOD-like receptors (NLRs), RIG-like receptors (RLRs), and some other receptor families Nikayla R. Thompson, John J. Kaminski, Evelyn A. Kurt-Jones, and Katherine A. Fitzgerald. Pattern Recognition Receptors and the Innate Immune Response to Viral Infection.//Viruses, 2011 June; 3(6): 920-940. Takeuchi O, Akira S. Pattern recognition receptors and inflammation.//Cell, 2010 Mar. 19; 140(6):805-201. Intracellular signaling pathways have been described in detail, beginning with the activation of PRRs and ending with the activation of NF-kB, AP-1, IRF, and other transcription factors that control the expression of certain genes and subsequent production of proteins encoded by these genes.

The below given examples 4, 5, 6, 7, 8, 9, and 10 illustrate that the enhancement of target transgene expression is achieved by activation of a wide range of PRRs, more specifically, members of the TLR family such as TLR2, TLR4, TLR5, TLR7, TLR8, and TLR9, and also members of the NLR family, specifically, NOD1- and NOD2-receptors.

The enhancement of target GFP expression is achieved by using the following natural agonists of PRRs or their synthetic analogues:

TLR2 ligands—lipoteichoic acid (LTA) and lipopeptide (Lipopeptide, Lot A11, EMC microcollections, GMBH);

TLR4 ligands—lipopolysaccharide (LPS) from E. coli serotype 055:B5 (Sigma L-2880) and acidic peptidoglycan having a molecular weight of 1200-40000 kilodaltons (APG, Russian Patent no. 2195308);

TLR5 ligand—flagellin (Invivogen);

TLR7 and TLR8 ligands—imiquimod and CL097 (imidazoquinoline derivative, Invivogen); and

TLR9 ligands—CpG-oligonucleotide ODN 1826 and ODN 2006 (Invivogen).

The example 4 illustrates that the enhancement of target transgene expression is achieved by activation of NOD-receptors using:

NOD1 ligands C12-iE-DAP (Lauroyl-g-D-Glu-D-mDAP and Lauroyl-g-D-Glu-L-mDAP, which are synthetic fragments of bacterial peptidoglycan, Invivogen) and

NOD2 ligand L18-MDP (derivative of muramyl dipeptide, which is a synthetic fragment of bacterial peptidoglycan, Invivogen).

The specific feature of the present invention is the possibility to use the effect of enhanced production of target transgene in vivo even, most notably, with the use of pharmaceutical agonists of PRRs.

The example 10 illustrates the enhancement of target protein expression by using pharmaceutical agonists of the following receptors:

agonists of TLR4, specifically, Immunomax (Reg. no. 001919/02-171011, Immapharma, Russia) and Pyrogenalum (Reg. no. 003478/0, Medgamal, Gamaleya Institute of Epidemiology and Microbiology, Russia) and

agonist of NOD2 receptors, specifically, Licopid (Reg. no. 001438, ZAO PEPTEK, Russia).

The enhancement of target protein expression is demonstrated by the use of not only PRR agonists but also cytokines, i.e. tumor necrosis factor (TNF-α, example 6).

The examples 6, 7, and 8 illustrate the enhancement of secretory protein expression by using a DNA vector with inserted SEAP gene.

The example 9 illustrates the enhancement of target membrane protein expression by using DNA vectors with inserted hemagglutinin genes of influenza H1N1, H3N2, or B viruses.

The implementation of the present invention was demonstrated in mouse (examples 4, 5, 6, 7, 8, and 10) and human (example 9) cells. Example 8 illustrates the enhancement of transgene expression and target protein production in vivo, specifically, in laboratory mice, although this example does not limit a use of the present invention in other animals and humans.

The following examples of preferred embodiments of the present invention are given to explain the invention in greater detail.

### EXAMPLES OF HOW TO USE THE INVENTION

### Example 1

### Construction of Plasmids Encoding Cytoplasmic, Secretory, or Membrane Protein

As a DNA vector, replication-defective recombinant human adenovirus (serotype 5) nanoparticles are used.

At the first stage, to obtain RDRANs, plasmid constructs are created, which bear expression cassettes containing nucleotide sequences which encode cytoplasmic GFP, secreted SEAP protein, and HA1, HA3, or HA-B membrane proteins. Thus, plasmid constructs pShuttle-CMV-GFP, pShuttle-CMV-SEAP, pShuttle-CMV-HA1, pShuttle-CMV-HA3, and pShuttle-CMV-HA-B are obtained.

The pShuttle-CMV plasmid construct with the genome fragments of type 5 human adenovirus (AdEasy Adenoviral Vector System, Stratagene Cat. No. 240009), specifically designed for obtaining of replication-defective recombinant adenovirus nanoparticles, is used to obtain the following plasmid constructs: pShuttle-CMV-GFP, pShuttle-CMV-SEAP, pShuttle-CMV-HA1, pShuttle-CMV-HA3, and pShuttle-CMV-HA-B. The pShuttle-CMV plasmid construct is hydrolyzed at the EcoRV restriction endonuclease site, and then nucleotide sequences encoding GFP, SEAP, HA1, HA3, or HA-B protein are inserted, and the pShuttle-CMV-GFP, pShuttle-CMV-SEAP, pShuttle-CMV-HA1, pShuttle-CMV-HA3, or pShuttle-CMV-HA-B plasmid constructs, respectively, are obtained. Nucleotide sequences of cytoplasmic GFP, secreted SEAP protein, and HA1, HA3, and HA-B membrane proteins to be inserted in pShuttle-CMV are obtained by hydrolyzing the corresponding plasmid constructs pGREEN (USA, Carolina Biological Supply Company), pAL-SEAP, pAL-HA1, pAL-HA3, and pAL-HA-B (chemical synthesis, Russia, Evrogen) at the sites of Ase I (pGREEN) and EcoRV (pAL-SEAP, pAL-HA1, pAL-HA3, and pAL-HA-B) restriction endonucleases. The presence of GFP, SEAP, HA1, HA3, and HA-B protein genes in the corresponding plasmid constructs pShuttle-CMV-GFP, pShuttle-CMV-SEAP, pShuttle-CMV-HA1, pShuttle-CMV-HA3, and pShuttle-CMV-HA-B is confirmed by the restriction analysis using EcoRI, NotI, and EcoRV endonucleases and by PCR.

This is the way plasmid constructs pShuttle-CMV-GFP, pShuttle-CMV-SEAP, pShuttle-CMV-HA1, pShuttle-CMV-HA3, and pShuttle-CMV-HA-B are obtained, which bear expression cassettes containing nucleotide sequences encoding cytoplasmic GFP, secreted SEAP protein, and HA1, HA3, or HA-B membrane proteins that are further used for obtaining of RDRANs.

### Example 2

### Obtaining and Testing of Replication-Defective Recombinant Adenovirus Nanoparticles with Inserted Genes of Target Proteins GFP, SEAP, HA1, HA3, or HA-B

Replication-defective recombinant adenovirus nanoparticles Ad-GFP, Ad-SEAP, Ad-HA1, Ad-HA3, and Ad-HA-B, which bear expression cassettes containing nucleotide sequences encoding cytoplasmic GFP, secreted SEAP protein, and HA1, HA3, and HA-B membrane proteins, respectively, are obtained using the AdEasy Adenoviral Vector System (Stratagene, Cat. No 240009) via homologous recombination of adenoviral genome fragments in E. coli cells. The presence of GFP, SEAP, HA1, HA3 HA-B protein genes in RDRANs is confirmed by PCR. Then titers of replication-defective recombinant adenovirus nanoparticles Ad-GFP, Ad-SEAP, Ad-HA1, Ad-HA3, and Ad-HA-B are determined by the plaque formation assay in HEK293 (human embryonic kidney cells) cell culture [Graham F. L., Prevec L. Manipulation of adenovirus vectors.//Methods in Mol. Biol., 1991, v. 7, p. 109-127].

The expression of obtained DNA vectors and the production of corresponding target proteins were examined in A549 cell culture. The expression of cytoplasmic target protein GFP was measured using an inverted fluorescence microscope (ICM-405, Leitz) and by flow cytofluorometry using a FACSAria II (BD Biosciences).

The expression of secreted target protein SEAP was measured according to J. Berger et al. with minor modifications [Berger J, Hauber J, Hauber R, Geiger R, Cullen B R. Secreted placental alkaline phosphatase: a powerful new quantitative indicator of gene expression in eukaryotic cells.//Gene, 1988 Jun. 15; 66 (1):1-10]. A test liquid was clarified by centrifugation at 14,000 g for 2 min, warmed-up at 65° C. for 5 min to inhibit endogenous phosphatases, then 20 μl aliquots were transferred into each well of a 96-well plate to 130 μl of reaction buffer (0.5 M NaHCO3, 0.5 MM MgCl₂, pH 9.8), and incubated at 37° C. for 10 min. Then 50 μl of 60 μM p-nitrophenyl phosphate in reaction buffer were added, and the optical density was measured at 405 nm at fixed intervals. The SEAP activity was expressed in mU/ml, considering 1 mU/ml corresponds to an increase in optical density of 0.04 U per 1 min.

The expression of target membrane proteins HA1, HA3, and HA-B was measured by staining cells with anti-HA1, anti-HA3, and anti-HA-B monoclonal antibodies, respectively, with a following flow cytofluorometry of stained cells using FACSAria II (BD Biosciences).

This is the way the DNA vectors, RDRANs (Ad-GFP, Ad-SEAP, Ad-HA1, Ad-HA3, and Ad-HA-B) bearing express cassettes containing nucleotide sequences which encode cytoplasmic GFP, secreted SEAP protein, and HA1, HA3, or HA-B membrane proteins, respectively, were obtained, and the expression of corresponding proteins was confirmed.

### Example 3

### Acidic Peptidoglycan (APG) Having a Molecular Weight of 1200-40000 kDa (Russian Patent No. 2195308) and a Pharmaceutical Drug Immunomax (Reg. No. 001919/02) are TLR4 Agonists

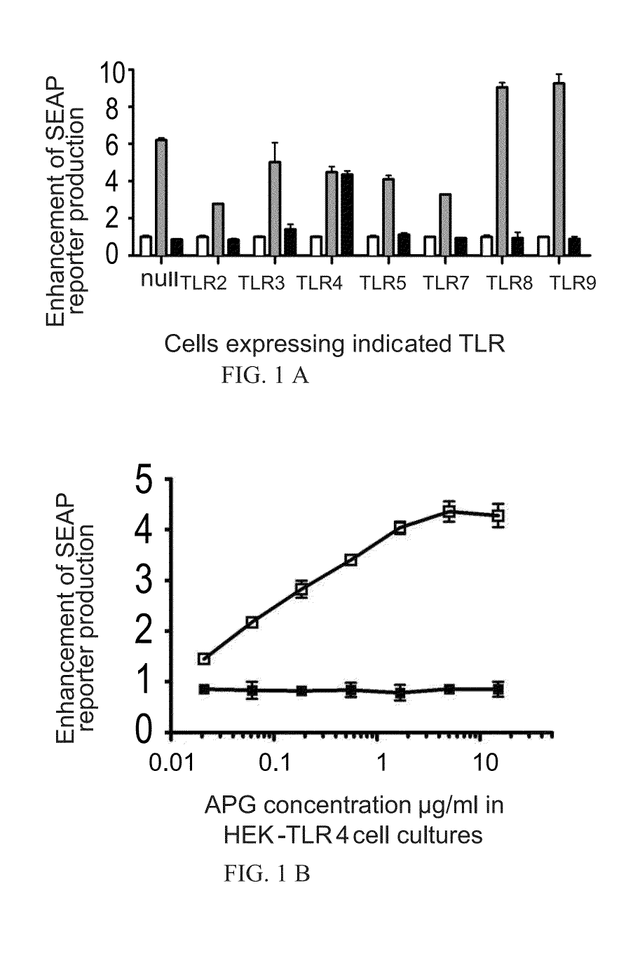

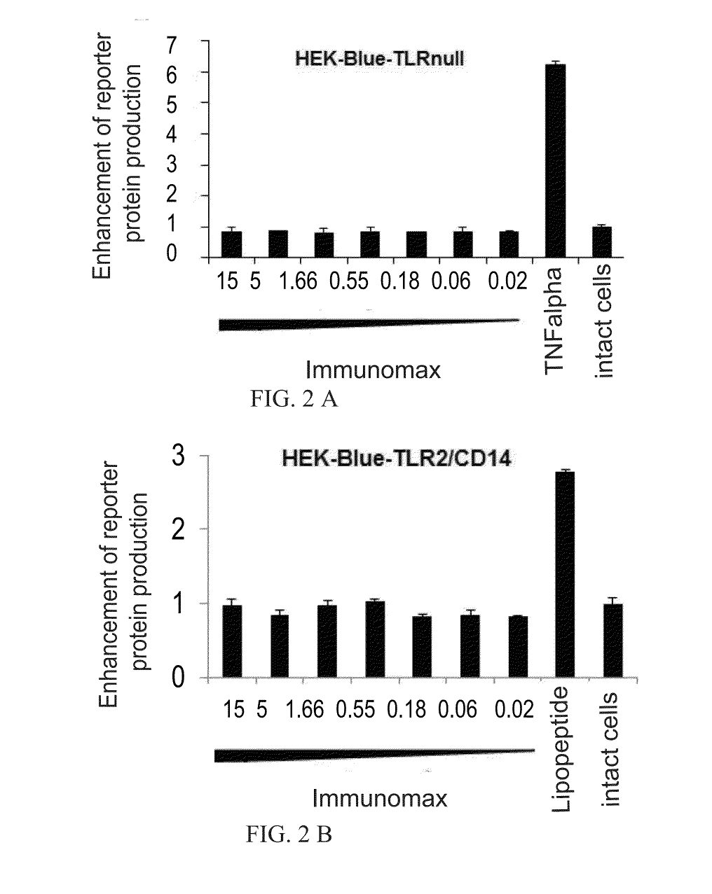

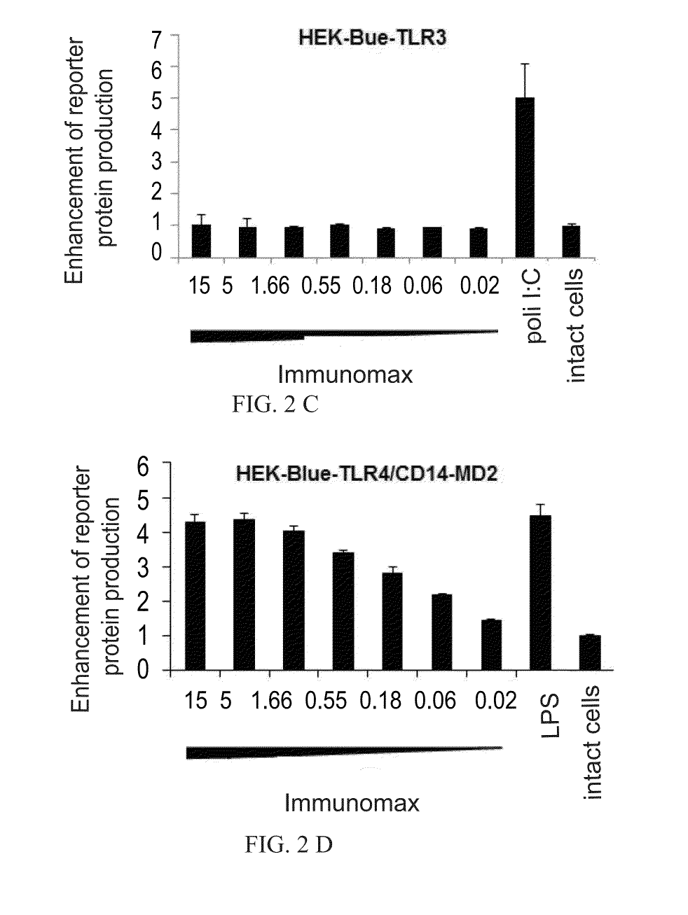

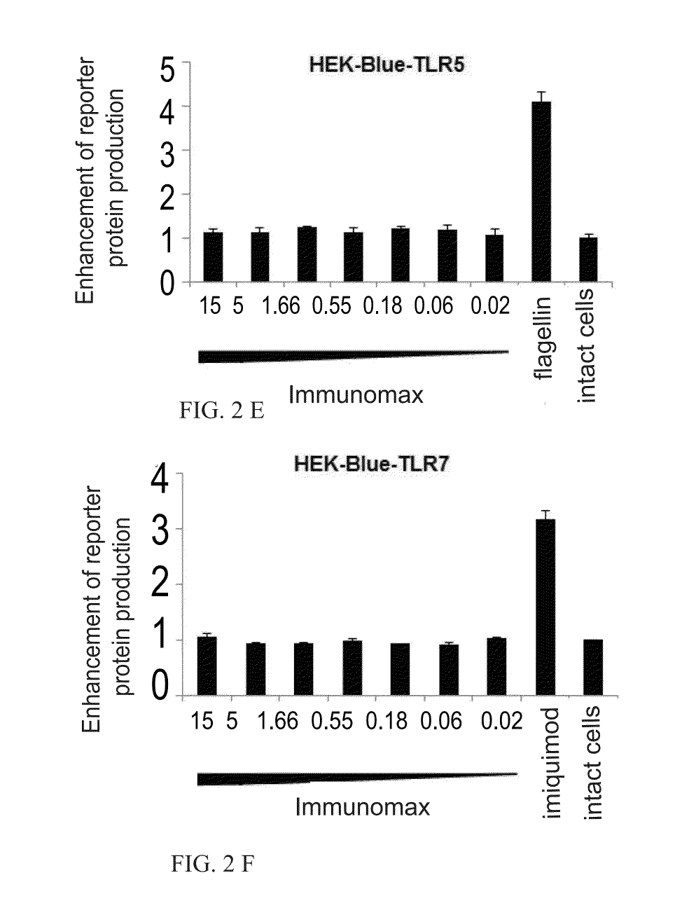

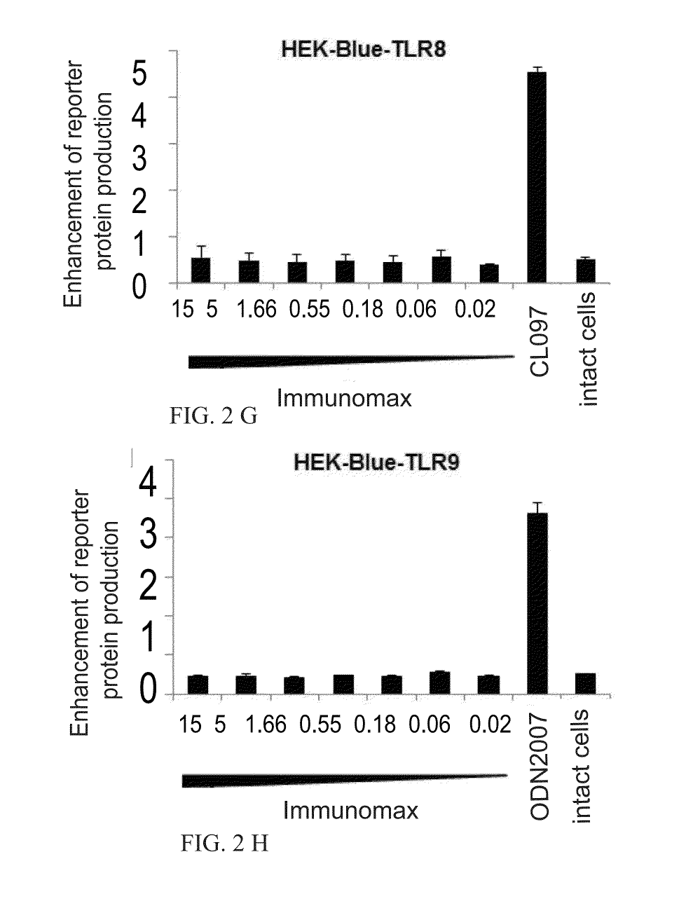

To identify cellular receptors for APG ((Russian Patent no. 2195308) and Immunomax (Reg. No. 001919/02), a collection of HEK-Blue (InvivoGen) cell lines was used, which cells stably express either TLR2, TLR3, TLR4, TLR5, TLR7, TLR8, or TLR9. All of the used HEK-Blue cell lines have an inducible SEAP reporter gene controlled by an NF-kB dependent promoter. In this cell lines, a signal from TLR leads to the secretion of SEAP reporter protein into the culture medium. The study using HEK-Blue cells expressing different TLRs showed that APG (Russian Patent no. 2195308) and Immunomax, a pharmaceutical compound, are TLR4 agonists. Both effectors activated NF-kB dependent production of SEAP reporter protein only in cells expressing TLR4 receptors (FIG. 1A, FIG. 2). Activation of TLR4-NF-kB signaling pathway directly depended on the agonist concentration (FIG. 1B, FIG. 2).

The above data confirm that both APG (Russian Patent no. 2195308) and Immunomax, a registered pharmaceutical drug (Reg. no. 001919/02) are TLR4 agonists.

### Example 4

### Enhancing Expression of Target Cytoplasmic GFP in Mouse Peritoneal Macrophages In Vitro by Using Agonists of PRRs, Members of the TLR and NOD Receptor Families

Mouse peritoneal macrophages (BALB/c female mice, Stolbovaya breeding nursery) were washed with physiological saline solution, pelleted by centrifugation at 1200 rpm for 10 min, suspended in the complete cell culture medium (CCM, consisting of RPMI-1640 medium supplemented with 10% fetal calf serum, 2 mM L-glutamine, 50 μM of (3-mercaptoethanol, and 10 μg/ml of gentamycin) at a concentration of one million cells per 1 ml, and incubated over night in 90 mm Petri dishes at 37° C. and 5% CO2. On the next day, nonadherent cells were removed by washing with warm phosphate buffered saline (PBS, pH 7.4), and adherent cells were covered with Versene solution, kept at 4° C. for 60 min in a refrigerator, and then detached from the culture dish by washing with Versene solution using 5 ml pipette. Macrophages were pelleted by centrifugation at 1200 rpm for 10 min, suspended in the CCM, and incubated in a 96-well plate at a concentration of 0.1 million/ml in a volume of 200 μl per well in triplets with addition of 2×10⁷ PFU/ml Ad-GFP (obtained as in the example 2) in combination with TLR or NOD-receptor agonists or without them (control). Cell cultures were incubated for 4 days at 37° C. and 5% CO₂. Table 1 shows the used TLR and NOD receptor agonists, and their final concentration in vitro.

TABLE 1
PRR agonists used in the example 4

| | | Final |
|---|---|---|
| Receptor | Agonist (ligand) | concentration |
| TLR2 | Lipoteichoic acid (Invivogen) | 10 μg/ml |
| TLR2 | Lipopeptide | 1 μg/ml |
| | (EMC microcollections, GMBH) | |
| TLR4 | E.coli lipopolysaccharide | 10 μg/ml |
| | (Invivogen) | |
| TLR4 | APG (Russian Patent no. | 10 μg/ml |
| | 2195308) | |
| TLR5 | Flagellin (Invivogen) | 1 μg/ml |
| TLR7/8 | Imiquimod (Invivogen) | 10 μg/ml |
| TLR7/8 | CL097 (Invivogen) | 2.5 μg/ml |
| TLR9 | ODN 1826 (Invivogen) | 0.6 μg/ml |
| TLR9 | ODN 2006 (Invivogen) | 10 μg/ml |
| NOD1 | C12-iE-DAP (Invivogen) | 20 μg/ml |
| NOD2 | L18-MDP (Invivogen) | 20 μg/ml |

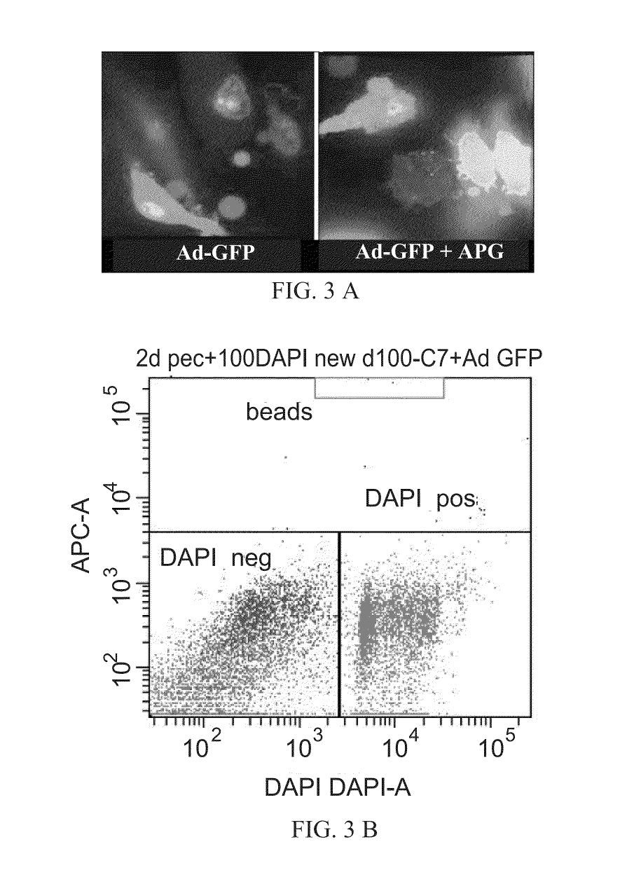

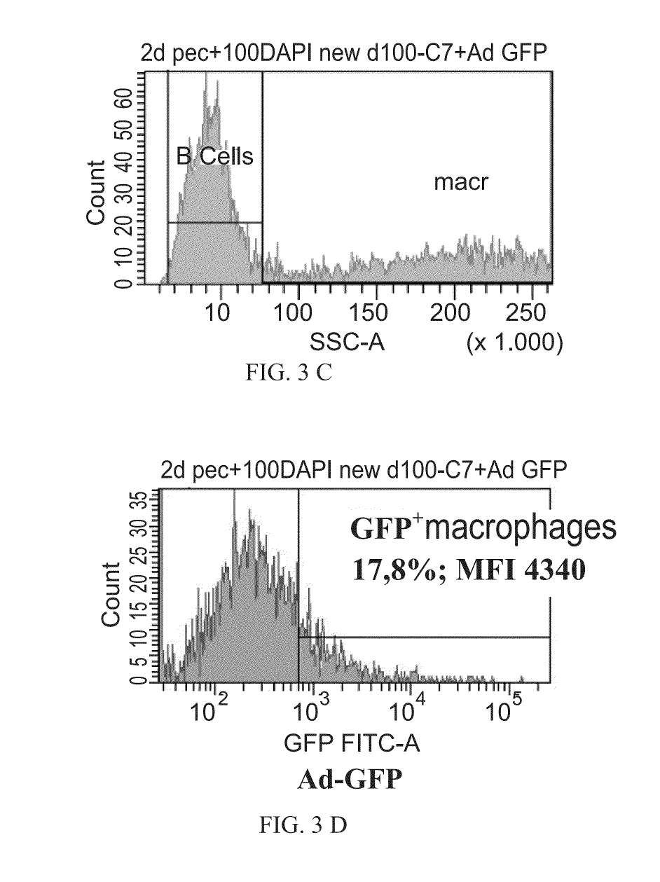

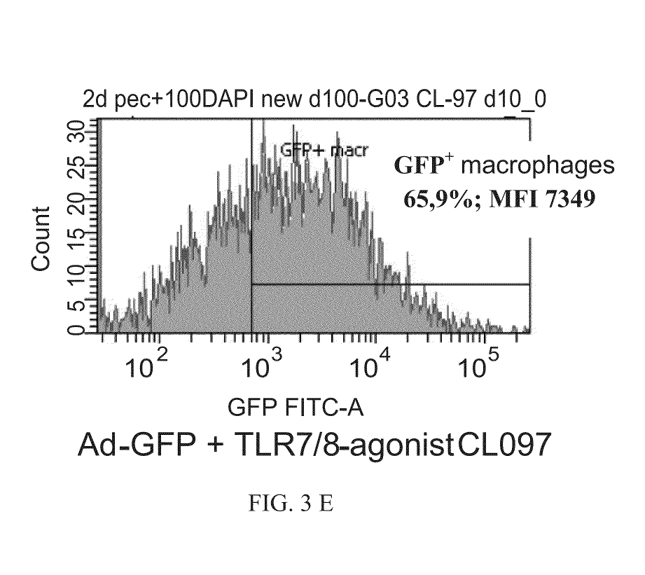

Confocal microscopy images (FIG. 3A) show macrophages transduced with Ad-GFP and additionally activated by TLR4 agonist (10 μg/ml of APG, right image), as compared to control macrophages transduced with Ad-GFP without addition of APG (left image). The enhancement of fluorescence intensity can be clearly seen in cells additionally stimulated by APG, evidencing the enhancement of synthesis of target GFP. The quantitative assay of GFP production was carried out using flow cytometry which determined both the absolute number of cells containing GFP and the GFP content in each cell by measuring fluorescence intensity of a single cell. The product of the number of fluorescent cells and the fluorescence intensity of each cell is proportional to the number of synthesized GFP molecules.

As is described earlier, peritoneal macrophages were transduced with Ad-GFP (2×10⁷ PFU/ml) in the absence or presence of one of the above mentioned PRR agonists. Cell cultures were incubated at 37° C. and 5% CO₂ for four days. After the end of incubation, cells were detached by washing with a cold Versene solution, and the percentage and absolute number of cells having green fluorescence and the intensity of cell fluorescence were determined using a FACSAria II flow cytometer. The absolute count of cells was normalized by calibration beads containing a known concentration of microspheres (CountBright Invitrogen), and discrimination between live and dead cells was carried out using a DNA-specific dye DAPI. Stages of cytometric discrimination of live cells and macrophages are shown in FIG. 3B and FIG. 3C. Typical histograms of GFP fluorescence signal after the transduction of macrophages by Ad-GFP without additional activation of cells (FIG. 3D) or with additional activation of cells with CL097, the agonist of TLR7 and TLR8 (FIG. 3E), are shown.

The quantitative assessment of the enhancement of target GFP expression was performed by the formula:

K=MFI×N/MFI_{contr}×N_{contr}, (1)

where

K is the enhancement (fold increase) of GFP expression;

MFI is the mean intensity of fluorescence of cells transduced with Ad-GFP with additional activation by an agonist;

MFI_{contr} is the mean intensity of fluorescence of cells transduced with Ad-GFP (control) without additional activation;

N is the number of fluorescent cells after the transduction with Ad-GFP with additional activation by an agonist; and

N_{contr} is the number of fluorescent cells (control) after the transduction with Ad-GFP without additional activation.

The data given in Table 2 show that the enhancement of GFP expression in peritoneal macrophages transduced with Ad-GFP was observed with different agonists of PRRs, members of the TLR and NOD receptor families, specifically, agonists of TLR2 (lipopeptide, lipoteichoic acid), TLR4 (APG, lipopolysaccharide), TLR5 (Flagellin), TLR7/8 (CL097, Imiquimod), TLR9 (CpG 2006, ODN 1826), NOD-1 receptors (C12-iE-DAP), and NOD-2 receptors (L18-MDP).

TABLE 2
Enhancement of GFP expression in mouse peritoneal
macrophages transduced with Ad-GFP (2 × 107 PFU/ml)
and activated by the indicated PRR agonist

| | | Fold increase in the | | |
|---|---|---|---|---|
| | | expression of GFP, | | |
| | | calculated according | | |
| | | to the formula (1) | | |
| | | | Standard | |
| PRR | Agonist | Mean | deviation | P |
| TLR2 | Lipopeptide, | 7.5 | 1.3 | <0.05 |
| | 10 μg/ml | | | |
| TLR2 | Lipopeptide, | 16.6 | 3.2 | <0.05 |
| | 1 μg/ml | | | |
| TLR2 | LTA, | 2.10 | 0.12 | <0.05 |
| | 10 μg/ml | | | |
| TLR4 | APG, | 10.5 | 2.2 | <0.05 |
| | 1.5 μg/ml | | | |
| TLR4 | LPS, | 1.96 | 0.08 | <0.05 |
| | 10 μg/ml | | | |
| TLR5 | Flagellin, | 2.13 | 0.18 | <0.05 |
| | 1 μg/ml | | | |
| TLR7/8 | CL097, | 14.2 | 1.6 | <0.05 |
| | 2.5 μg/ml | | | |
| TLR7/8 | Imiquimod, | 2.03 | 0.25 | <0.05 |
| | 10 μg/ml, | | | |
| TLR9 | CpG 2006, | 3.1 | 0.6 | <0.05 |
| | 10 μg/ml | | | |
| TLR9 | ODN 1826, | 1.43 | 0.19 | <0.05 |
| | 0.6 μg/ml | | | |
| NOD1 | C12-iE-DAP, | 3.1 | 1.3 | <0.05 |
| | 20 μg/ml | | | |
| NOD2 | L18-MDP, | 3.7 | 1.2 | <0.05 |
| | 2 μg/ml | | | |

### Example 5

### Expression Enhancement of a Target Cytoplasmic Protein in Mouse Bone Marrow-Derived Dendritic Cells In Vitro by Using a TLR4 Agonist

Mouse bone marrow cells (BALB/c female mice, Stolbovaya breeding nursery) were washed from the femur and tibia with physiological saline; erythrocytes were lysed by hypotonic shock using distilled water for 15 min, osmoticity was then immediately restored by adding the required amount of 10× Hanks' balanced salt solution, cells were pelleted by centrifugation at 1200 rpm for 10 min, re-suspended in the CCM at a concentration of one million cells per 1 ml, and incubated in 90 mm Petri dishes in the presence of 20 ng/ml GM-CSF. After 3-4 days, the culture medium was replaced by a fresh CCM supplemented with GM-CSF. After 7 days, nonadherent cells were collected, suspended in the CCM, and incubated in a 96-well plate at a concentration of 0.1 million/ml in a volume of 200 μl per well in triplets with addition of 2×10⁷ PFU/ml Ad-GFP (obtained as in the example 2), with or without 5 μg/ml of APG (Russian Patent no. 2195308). Two days after the addition of Ad-GFP, cells were collected by washing with Versene solution, and the percentage of fluorescent cells was determined using a FACSAria II flow cytometer. Results are shown in Table 3. The expression of target GFP in bone marrow-derived dendritic cells was increased five-fold under activation of these cells with APG (Russian Patent no. 2195308).

TABLE 3
Enhancement of transgene expression in bone marrow dendritic cells under
activation with a TLR4 agonist (APG, Russian Patent no. 2195308)

| | Percentage of dendritic cells expressing GFP | | |
|---|---|---|---|
| | | Standard | |
| Transduction | Mean | deviation | P |
| Ad-GFP 2 × 10⁷ PFU/ml | 3 | 0.5 | — |
| Ad-GFP 2 × 10⁷ PFU/ml + | 15 | 1 | 0.05 |
| APG (Russian patent no. | | | |
| 2195308), 5 μg/ml | | | |
| Enhancement coefficient | 5 | — | — |

### Example 6

### Enhancing Expression of a Target SEAP Protein in Mouse Peritoneal Macrophages In Vitro by Using a Cytokine TNF-α

Mouse peritoneal macrophages were obtained and incubated as described in the example 4. Cells were incubated in triplet culture wells with addition of Ad-GFP at 2×10⁷ PFU per well in combination with TNF-α (10 ng/ml) or APG (Russian Patent no. 2195308, 10 μg/ml). In the negative control cultures, macrophages were transduced with Ad-SEAP (2×10⁷ PFU) without any cell activating compound. After 4 days, the activity of SEAP was measured in the culture medium, as described in the example 2. The SEAP expression was increased 1.8-fold when macrophages were activated with TNF-α (Table 4).

TABLE 4
Enhancement of expression of transgenic SEAP protein
in peritoneal macrophages under their activation
with a cytokine TNF-α or a TLR4 agonist APG

| | Activity of SEAP (mU/ml) | | |
|---|---|---|---|
| | in culture supernatant | | |
| | | Standard | |
| Transduction | Mean | deviation | P |
| Ad-SEAP (2 × 10⁷ PFU per well) | 0.34 | 0.06 | — |
| Ad-SEAP 2 × 10⁷ PFU per well + TNF-α | 0.62 | 0.12 | <0.05 |
| (10 ng/ml) | | | |
| Ad-SEAP (2 × 10⁷ PFU per well) + APG | 0.82 | 0.03 | <0.05 |
| (Russian patent no. 2195308, 10 μg/ml) | | | |
| Enhancement coefficient for TNF-α | 1.8 | — | — |
| Enhancement coefficient for APG | 2.4 | — | — |
| (Russian patent no. 2195308) | | | |

### Example 7

### Enhancing Target SEAP Protein Expression in Mouse Bone Marrow-Derived Dendritic Cells In Vitro by Using a TLR4 Agonist

Mouse bone marrow-derived dendritic cells were obtained as described in the example 5. The suspension of dendritic cells in the CCM was incubated in a 96-well plate at a concentration of 0.1 million/ml in a volume of 200 μl per well in triplets with the addition of 2×10⁷ PFU/ml Ad-GFP, obtained as in the example 2, with or without APG (Russian Patent no. 2195308, 5 μg/ml). After 6 days, the activity of SEAP was measured in the culture medium, as described in the example 2. Upon activation by a TLR4 agonist (APG, Russian Patent no. 2195308), the SEAP expression in dendritic cells was increased six-fold (Table 5).

TABLE 5
Enhancement of expression of transgenic SEAP protein in dendritic
cells in vitro under their activation with a TLR4 agonist

| | Activity of SEAP (mU/ml) | | |
|---|---|---|---|
| | in culture supernatant | | |
| | | Standard | |
| Transduction | Mean | deviation | P |
| Ad-SEAP (2 × 10⁷ PFU) | 0.48 | 0.07 | — |
| Ad-SEAP (2 × 10⁷ PFU) + | 3.0 | 0.3 | <0.05 |
| APG (Russian patent no. 2195308, | | | |
| 5 μg/ml) | | | |
| Enhancement coefficient | 6 | — | — |

### Example 8

### Enhancing Transgene Expression of SEAP Protein in Laboratory Mice In Vivo by Using a Pharmaceutical Agonist of TLR4 (Immunomax)

Ad-SEAP was obtained as described in the example 2 and was injected at a dose of 10⁸ PFU per mouse in 200 μl of physiological saline into mouse peritoneal cavity (BALB/c female mice, 14-16 g, Stolbovaya breeding nursery of the Russian Academy of Medical Sciences) in combination with 10 μg of Immunomax (Immapharma, Russia) or without it. After 3 days, blood samples were collected from the retro-orbital sinus of each mouse, then blood serum was prepared, in which the SEAP activity was measured as described in the example 2 Immunomax caused a seven-fold increase in the SEAP expression (Table 6). None of adverse (toxic) effects were observed in animals injected with Immunomax in combination with Ad-SEAP. This result opens a possibility of enhancing transgene expression in animals and human subjects by using Immunomax, a pharmaceutical agonist of TLR4.

TABLE 6
Enhancement of SEAP transgene expression in laboratory mice in vivo by
using a single injection of pharmaceutical TLR4- agonist (Immunomax)

| | Activity of SEAP in blood | | |
|---|---|---|---|
| | serum, mU/ml | | |
| | | Standard | |
| Transduction in vivo | Mean | deviation | P |
| Ad-SEAP (10⁸ PFU per mouse) | 4.35 | 4.69 | — |
| Ad-SEAP (10⁸ PFU per mouse) + | 30.42 | 12.47 | 0.02 |
| Immunomax 10 μg per mouse | | | |
| Enhancement coefficient | 7 | — | — |

### Example 9

### Enhancing Transgene Expression Encoding Membrane Proteins HA1, HA3, and HA-B in Human Cell Cultures In Vitro by Using a TLR4 Agonist

Blood samples were obtained from healthy donors by cubital vein puncture and collected into BD Vacutainer tubes with K₃EDTA; for isolating the mononuclear cell fraction, blood samples were diluted 1:2 in physiological saline, layered over Ficoll (1.077 g/l, PanEco, Russia), and centrifuged at 400 g and 20° C. for 25 min The fraction containing mononuclear cells was collected into a 15-ml tube; cells were washed in PBS (supplemented with 0.5% BSA, 1% glucose, and 10 mM HEPES, pH 7.4), re-suspended at a concentration of 3 million per 1 ml of CCM and dispensed at 0.5 ml per well in duplicate wells of Nunclon 24-well plate. Then one of Ad-HA1, Ad-HA3, or Ad-HA-B (5×10⁶ PFU) vectors was added in cell cultures with 5 μg/ml of APG (RU no. 2195308) or without it. The plate was incubated at 37° C. and 5% CO₂ for 2 days. After 2 days, cells were removed from wells using a Versene solution, transferred to 15-ml tubes, and pelleted by centrifugation. Cell pellet was stained with monoclonal antibodies against HA1, HA3, or HA-B (SinoBiological) for 20 min and washed with PBS (supplemented with 0.5% BSA, 0.01% sodium azide, 0.35 mM EDTA, and 10 mM HEPES, pH 7.4). Primary antibodies bound to HA were detected with FITC labeled Fab-fragments of anti-mouse IgG. Cells were washed with PBS (supplemented with 0.5% BSA, 0.01% sodium azide, 0.35 mM EDTA, and 10 mM HEPES, pH 7.4); monocytes were stained with CD14 PE-Cy7 antibodies (BD Biosciences). The percentage of cells expressing HA among the population of CD14-positive cells was determined using a FACSAria II flow cytometer. After activation of cells with APG (Russian Patent no. 195308), expression of target membrane proteins HA1, HA3 HA-B was increased 1.75- to 4.1-fold (Table 7).

TABLE 7
Enhancement of HA1, HA3, and HA-B expression after the
activation with a TLR4 agonist of human blood mononuclear cells,
which are transduced with Ad-HA1, Ad-HA3, or Ad-HA-B vectors

| | Coefficient of increase | | |
|---|---|---|---|
| | in the percentage of | | |
| | monocytes expressing HA* | | |
| | | Standard | |
| Transduction | Mean | deviation | P |
| Ad-HA1 (5 × 10⁶ PFU) + APG | 1.75 | 0.26 | <0.05 |
| (Russian patent no. 2195308), 5 | | | |
| MκΓ/MJI | | | |
| Ad-HA3 (5 × 10⁶ PFU) +APG | 3.2 | 0.12 | <0.05 |
| (Russian patent no. 2195308), 5 | | | |
| μg/ml | | | |
| Ad-HA-B (5 × 10⁶ PFU) + APG | 4.1 | 1.04 | <0.05 |
| (Russian patent no. 2195308), 5 | | | |
| μg/ml | | | |

*Note:
Data were normalized by the percent of cells expressing the corresponding HA in control cell cultures without APG.

### Example 10

### Enhancing Transgene Expression Encoding Cytoplasmic GFP in Mouse Peritoneal Macrophages In Vitro by Using Pharmaceutical Agonists of PRRs, Members of the TLR and NOD Receptor Families

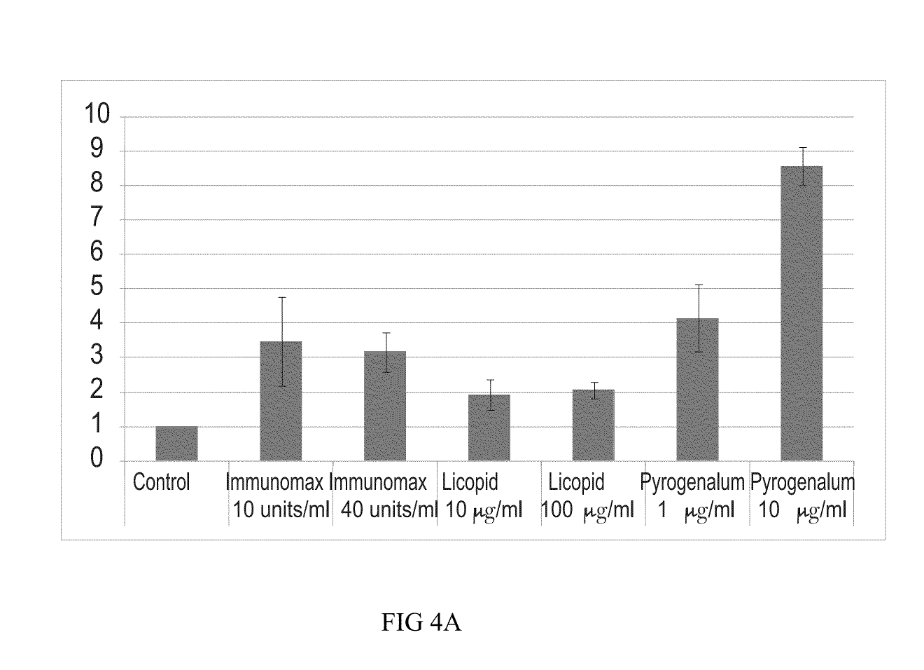

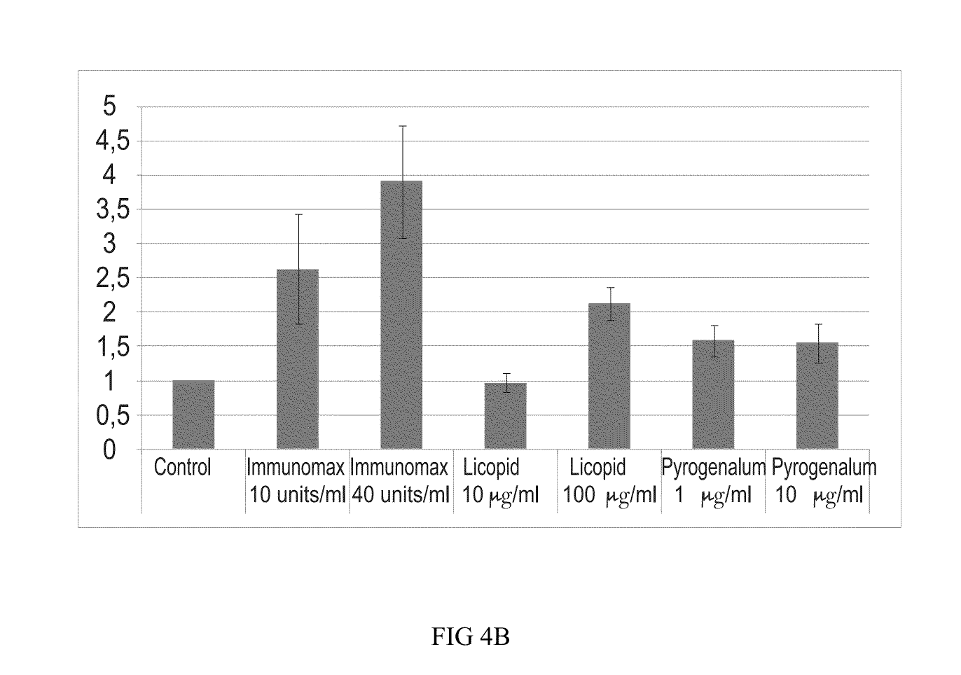

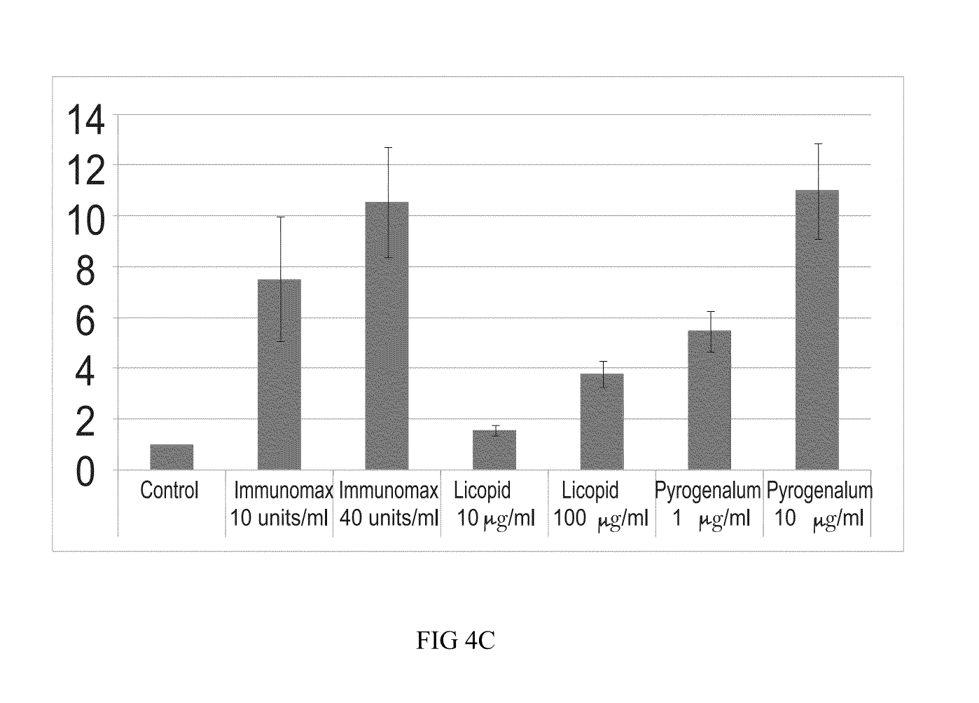

Mouse peritoneal macrophages were obtained and incubated as described in the example 4. Cells were incubated in triplets and transduced with Ad-GFP vector at 5×10⁶ PFU/ml. To activate macrophages, a pharmaceutical agonist of TLR4 receptors, specifically, Immunomax (Reg. no. 001919/02-171011, Immapharma, Russia), or Pyrogenalum (Reg. no. 003478/0, Medgamal, Gamaleya Institute of Epidemiology and Microbiology, Russia), or a pharmaceutical agonist of NOD receptors Licopid (Reg. no. 001438, ZAO PEPTEK, Russia), was added into cell cultures. Cultures of macrophages transduced with Ad-GFP without activators served as controls. After 2 days, the expression of target protein was assessed as described in the example 4. Results of experiments are shown in FIG. 4. They prove that all the drugs used, i.e., pharmaceutical agonists of PRRs cause a significant enhancement of target transgene expression. After stimulation of cells with pharmaceutical agonists of PRRs, production of target proteins was increased 2- to 11-fold. The efficacy of enhancement depended on the dose of agonist. This result means that the transgene expression may be enhanced in humans by using pharmaceuticals which activate transduced cells via PRRs.

### INDUSTRIAL APPLICABILITY

All above mentioned examples confirm that the composition is industrially applicable and the technical task, namely to develop a composition for enhancing transgene expression in eukaryotic cells and a method for enhancing production of a target protein encoded by a transgene and to provide an opportunity of using the said composition and method both in cell culture in vitro and in a living body (in vivo), has been realized in the present invention.

### LIST OF ABBREVIATIONS

PRRs—pattern recognition receptors, cell receptors for recognition of molecular patterns associated with microbial pathogens, viruses, cellular stress and damage
TLRs—Toll-like receptors, cell receptors related to PRRs
NOD receptors—nucleotide-binding oligomerization domain-containing receptors, cell receptors related to PRRs
NLRs—NOD-like receptors related to PRRs
RLR—RIG-like receptors related to PRRs
PAMP—pathogen associated molecular patterns, molecular patterns associated with microbial pathogens and viruses
DAMP—damage-associated molecular patterns, molecular patterns associated with damage
RDRANs—replication-defective recombinant adenovirus nanoparticles
GM-CSF—granulocyte-macrophage colony-stimulating factor
GFP—green fluorescent protein
Ad—replication-defective recombinant adenovirus (type 5)-based nanoparticles
Ad-GFP—RDRANs with inserted GFP gene
HA1—Influenza virus H1N1 hemagglutinin
HA3—Influenza virus H3N2 hemagglutinin
HA-B—Influenza virus B hemagglutinin
Ad-HA1—RDRANs with inserted HA1 gene
Ad-HA3—RDRANs with inserted HA3 gene
Ad-HA-B: RDRANs with inserted HA-B gene
SEAP—secreted embryonic alkaline phosphatase
Ad-SEAP—RDRANs with inserted SEAP gene
LTA—lipoteichoic acid, a component of bacterial wall
LPS—lipopolysaccharide, a component of bacterial wall
APG—acidic peptidoglycan
CL097—imidazoquinoline derivative
ODN—oligonucleotide
C12-iE-DAP—lauroyl-g-D-Glu-D-mDAP and lauroyl-g-D-Glu-L-mDAP, synthetic fragments of bacterial peptidoglycan
L18-MDP—derivative of muramyl dipeptide
PCR—polymerase chain reaction
CCM—complete cell culture medium
PBS—phosphate buffered solution
PFU—plaque forming units
TNF—tumor necrosis factor
BSA—bovine serum albumin
FITC—fluorescein isothiocyanate
EDTA—ethylenediaminetetraacetate
FACS—fluorescence-activated cell sorter (flow cytofluorimeter)
kDa—kilodalton

### REFERENCES

1. Dorokhov Y L, Skulachev M V, Ivanov P A, Zvereva S D, Tjulkina L G, Merits A, Gleba Y Y, Hohn T, Atabekov J G. Polypurine (A)-rich sequences promote cross-kingdom conservation of internal ribosome entry//Proc. Natl. Acad. Sci. USA, 2002, v. 99, p. 5301-5306.
2. Lee Y B, Glover C P, Cosgrave A S, Bienemann A, Uney J B. Optimizing regulatable gene expression using adenoviral vectors.//Exp Physiol., 2005 January; 90(1):33-37.
3. Li Z L, Tian P X, Xue W J, Wu J. Co-expression of sCD40LIg and CTLA4Ig mediated by adenovirus prolonged mouse skin allograft survival.—J Zhejiang Univ Sci B, 2006 June; 7(6):436-44.
4. US 20080241883 A1 Recombinant expression vector elements (rEVEs) for enhancing expression of recombinant proteins in host cells.
5. WO2008000445 Expression vector(s) for enhanced expression of a protein of interest in eukaryotic or prokaryotic host cells.
6. Siavoshian S., J-P Segain, M Kornprobst, C Bonnet, C Cherbut, J-P Galmiche, H M Blottiere. Butyrate and trichostatin A effects on the proliferation/differentiation of human intestinal epithelial cells: induction of cyclin D3 and p21 expression.//Gut, 2000; 46:507-514.
7. RU2443779 Method for obtaining recombinant adenovirus preparation characterized by a decreased ratio of physical and infectious viral particles and a gene therapy drug obtained using this method.
8. US20080311095 Methods and compositions for increased transgene expression (invention prototype).
9. Mikayla R. Thompson, John J. Kaminski, Evelyn A. Kurt-Jones, and Katherine A. Fitzgerald. Pattern Recognition Receptors and the Innate Immune Response to Viral Infection.//Viruses, 2011 June; 3(6): 920-940.
10. Takeuchi O, Akira S. Pattern recognition receptors and inflammation.//Cell, 2010 Mar. 19; 140(6): 805-20.
11. Graham F. L., Prevec L. Manipulation of adenovirus vectors.//Methods in Mol. Biol., 1991, v. 7, p. 109-127.
12. Berger J, Hauber J, Hauber R, Geiger R, Cullen B R. Secreted placental alkaline phosphatase: a powerful new quantitative indicator of gene expression in eukaryotic cells.//Gene, 1988 Jun. 15; 66 (1):1-10.
13. U.S. Pat. No. 6,019,978 Replication-defective adenovirus human type 5 recombinant as a vaccine carrier.

What is claimed is:

1. A composition for intensive production of target protein in eukaryotic cells, the composition comprising a DNA vector with an inserted gene of a target protein, and an agonist of cell receptors from a class of PRR—pattern recognition receptors, the antagonist being selected from the following agonists: TLR2 agonists, or TLR4 agonists, or TLR5 agonists, or TLR7 agonists, or TLR8 agonists, or TLR9 agonists, or NOD1 receptor agonists, or NOD2 receptor agonists, used in optimal ratios.

2. The composition according to claim 1, wherein the TLR2 agonist is a lipoteichoic acid.

3. The composition according to claim 1, wherein the TLR2 agonist is a lipopeptide.

4. The composition according to claim 1, wherein the TLR4 agonist is a bacterial lipopolysaccharide.

5. The composition according to claim 1, wherein the TLR4 agonist is an acidic peptidoglycan having a molecular weight of 1200-40000 kDa.

6. The composition according to claim 1, wherein the TLR5 agonist is a flagellin.

7. The composition according to claim 1, wherein the TLR7 agonist is an imiquimod.

8. The composition according to claim 1, wherein the TLR8 agonist is an imiquimod.

9. The composition according to claim 1, wherein the TLR7 agonist is an imidazoquinoline derivative CL097.

10. The composition according to claim 1, wherein the TLR8 agonist is an imidazoquinoline derivative CL097.

11. The composition according to claim 1, wherein the TLR9 agonist is an oligonucleotide CpG ODN 1826.

12. The composition according to claim 1, wherein the TLR9 agonist is an oligonucleotide CpG ODN 2006.

13. The composition according to claim 1, wherein the NOD1 receptor agonists is a C12-iE-DAP comprising Lauroyl-g-D-Glu-D-mDAP and Lauroyl-g-D-Glu-L-mDAP, synthetic fragments of bacterial peptidoglycan.

14. The composition according to claim 1, wherein the NOD2 receptor agonist is an L18-MDP—a derivative of a muramyl dipeptide, such as a bacterial peptidoglycan fragment.

15. The composition according to claim 1, wherein the PRR agonist is a pharmaceutical drug in an effective dose.

16. The composition according to claim 15, wherein the PRR agonist is Immunomax® (Registration No. 001919/02).

17. The composition according to claim 15, wherein the PRR agonist is Pyrogenalum® (Registration No. 003478/0).

18. The composition according to claim 15, wherein the PRR agonist is Licopid® (Registration No. 001438).

19. The composition according to claim 1, wherein the DNA-vector comprises replication-defective recombinant human adenovirus serotype 5 based nanoparticles.

20. The composition according to claim 1, wherein the DNA vector is a DNA-vector with an inserted target gene encoding a secretory protein.

21. The composition according to claim 1, wherein the DNA vector is a DNA-vector with an inserted target gene encoding a cytoplasmic protein.

22. The composition according to claim 1, wherein the DNA vector is a DNA-vector with an inserted target gene encoding a membrane protein.

23. A method comprising enhancing production of target protein encoded by a transgene in eukaryotic cells transduced with DNA vectors by using a composition for intensive production of target protein in eukaryotic cells, the composition comprising a DNA vector with an inserted gene of a target protein, and an agonist of cell receptors from a class of PRR—pattern recognition receptors, the antagonist being selected from the following agonists: TLR2 agonists, or TLR4 agonists, or TLR5 agonists, or TLR7 agonists, or TLR8 agonists, or TLR9 agonists, or NOD1 receptor agonists, or NOD2 receptor agonists, used in optimal ratios.

24. The method according to claim 23, wherein enhancing target protein production is achieved in eukaryotic cells in vitro.

25. The method according to claim 23, wherein enhancing of target protein production is achieved in vivo.

26. The method according to claim 23, wherein the eukaryotic cells are obtained from a mouse.

27. The method according to claim 23, wherein the eukaryotic cells are obtained from a human subject.

28. A composition for intensive production of target protein in eukaryotic cells, the composition comprising a DNA vector with an inserted gene of a target protein, and an agonist of cell receptors from a class of cytokine receptors, used in an optimal ratio.

29. The composition according to claim 28, wherein a tumor necrosis factor is used as a cytokine receptor agonist.

30. A method comprising enhancing production of target protein in eukaryotic cells transduced with DNA vectors by using the composition for intensive production of target protein in eukaryotic cells, the composition comprising a DNA vector with an inserted gene of a target protein, and an agonist of cell receptors from a class of cytokine receptors, used in an optimal ratio.

31. The method of claim 30, wherein a tumor necrosis factor is used as a cytokine receptor agonist.
